(19) United States
(12) Patent Application Publication
(10) Pub. No.: US 2007/0258941 A1
(43) Pub. Date: Nov. 8, 2007

(54) Methods and compositions for remediation of disc herniation by modifying structure

(76) Inventor: Brian E. Pfister, Wilmette, IL (US)

Correspondence Address: Sonia K. Guterman, Suite 345, 88 Black Falcon Avenue, Boston, MA, 02210-2481, US

(21) Appl. No.: 11/799,393

(22) Filed: May 1, 2007

Related U.S. Application Data
(60) Provisional application No. 60/796,719, filed on May 2, 2006.

(51) Int. Cl. A61K 31/785; A61K 31/715; A61K 31/718; A61K 31/722; A61K 38/47

(52) U.S. Cl. 424/78.27; 424/94.61; 514/55; 514/54; 514/59

(57) ABSTRACT

A therapeutic method and compositions are provided that involve injection of a cation solution into extracellular matrix of a tissue. In the preferred embodiment, a percutaneous (performed through the skin) injection of a polylysine solution into the nucleus of a herniated disc is provided, to reduce intradiscal pressure.

### RELATED APPLICATION

This application claims the benefit of provisional application 60/796,719 entitled, “Methods and compositions for remediation of disc herniation by modifying structure,” inventor Brian E. Pfister, filed in the U.S. Patent and Trademark Office May 2, 2006, which is hereby incorporated herein by reference in its entirety.

### TECHNICAL FIELD

The invention relates to methods and compositions for treatment and prevention of a cartilage disease, and repair and/or regeneration of a diseased or injured intervertebral disc.

### BACKGROUND

Low back pain is the primary cause of disability in individuals under the age of 40. The lifetime prevalence of low back pain in the population is about 70-85% with about 10-20% experiencing chronic low back pain (Andersson, G. B., Epidemiological features of chronic low-back pain. Lancet 354: 581-5, 1999), a major burden on the medical, social, and economic structures of essentially all countries. The costs for treatment of back pain in the United States are estimated to be 50 billion dollars per year.

Many patients who suffer with chronic low back pain have symptomatic bulging or herniated discs. Treatment for these conditions is limited to addressing symptoms, and the effectiveness of treatment is often questioned. Conventional treatment methods include medications, steroid injection, physical therapy, and surgery. Depending on the patient's symptoms and pathologic anatomy, surgical treatments may be simple outpatient microdiscectomy or a major surgical procedure such as fusion. Minimally invasive therapies for intervertebral disc (IVD) herniation such as endoscopic discectomy, percutaneous discectomy, and chemonucleolysis are available as well. However, clinical results of these procedures are not uniformly satisfactory, and these procedures may damage the disc structure, and may eventually lead to instability of the motion segment. Interbody fusion or other fusion procedures are often performed to stabilize the spinal motion segments. However, these procedures are associated with increased perioperative morbidities, and can result in decreased spinal mobility and adjacent segment degeneration.

### SUMMARY

An embodiment of the invention herein provides a pharmaceutical composition for remediating a cartilage disease, and the composition includes a cationic compound in an effective dose for remediating the disease, and a pharmaceutically acceptable salt or buffer. In related embodiments, the compound is a polymer of a cationic monomer.

For example, the compound is a polymer of an amino acid, and the amino acid is at least one selected from a group consisting of D-lysine, L-lysine, D-arginine, L-arginine, D-histidine, and L-histidine.

In various embodiments the compound is at least one selected from the group consisting of a dextran, an arabinogalactan, a pullulan, a cellulose, an inulin, a chitosan, an ornithine polymer, a spermine polymer, a spermidine polymer, and polyethylenimine. For example, the dextran is a branched poly-α-D-glucoside. Alternatively, the arabinogalactan comprises D-galactose and L-arabinose, and the arabinogalactan is produced by a plant, a fungus, or a bacterium. Alternatively, the pullulan comprises maltotriose, and the pullulan is produced by Aureobasidium pullulans. Alternatively, the inulin comprises fructosyl oligosaccharides. Alternatively, the chitosan is produced by at least one selected from the group consisting of: a fungus, an arthropod, or a marine invertebrate.

In certain embodiments, the polymer is at least about 2 kDa in average size. For example, the polymer is at least about 300 kDa in size. An exemplary polymer is a polylysine, comprising, for example, at least one of D-lysine and L-lysine monomers. Thus the polymer, for example the polylysine, is about 100 kDa to about 300 kDa in size. In general the polymer is of sufficient size to bind to a plurality of proteoglycan molecules. In certain embodiments, the composition includes a plurality of polycationic compounds selected for varying molecular weights, wherein a half-life of the compound in a cartilage is a function of the molecular weight.

In additional embodiments, the composition includes a growth factor or an enzyme inhibitor such as a matrix metalloproteinase inhibitor. For example, the growth factor is at least one growth factor selected from the group of: a bone morphogenesis protein such as BMP-2 and BMP-7, an insulin like growth factor (IGF), a transforming growth factor (TGF), a growth differentiation factor (GDF) such as GDF-5, and a platelet-derived growth factor (PDGF). In related embodiments, the growth factor or enzyme inhibitor is covalently bound to the compound.

Also provided herein is a method for remediating a cartilage disease in a subject, the method having the steps of: contacting the cartilage with a composition including a cationic compound, and observing remediation of the cartilage disease. In general, the cartilage is an intervertebral disc. In certain embodiments, the composition includes at least one of the group consisting of a dextran, an arabinogalactan, a pullulan, a cellulose, an inulin, a chitosan, an ornithine polymer, a spermine polymer, a spermidine polymer, and polyethylenimine. For example, the disc is herniated. Generally, contacting the cartilage is injecting a solution of the composition, for example, injecting is percutaneous such as intravertebral. In various related embodiments of the method, remediation is a reduction in at least one of disc height, back pain, sciatica, pinched nerve, extrusion, herniation, foot drop, and loss of ankle reflex. In certain embodiments, the remediation includes observing disc regeneration. In various related embodiments of the method, the compound is polylysine, for example, the polylysine includes L-lysine, D-lysine or the polylysine is a D- and L-heteropolymer.

In yet other embodiments, the invention provides a kit for treating a herniated disc, the kit having a cationic compound, a container, and instructions for use in treating a herniated disc. The kit can have the compound in a unit dose, for administration by hypodermic intravertebral injection.

### BRIEF DESCRIPTION OF THE DRAWINGS

FIG. 1 is a set of bar graphs showing intervertebral disc heights in rabbits injected with 30-70 kDa polylysine at each of lumbar 2/3 and 4/5, compared with heights of control untreated lumbar discs 3/4 (100%). Disc heights were measured at each of two weeks (FIG. 1, Panel A), four weeks (FIG. 1, Panel B), and six weeks (FIG. 1, Panel C). The data are shown in Table 1. A decrease in the DHI of approximately 20% was maintained for 6 weeks in this study.

FIG. 2 is a set of photographs of bovine cartilage explants that were incubated with FITC-labeled polylysine (15-30 kDa in size, panels F, G, H, I, and J; and 30-70 kDa in size, panels K, L, M, N, and O) or control untreated cartilage (FIG. 2 panels A, B, C, D, and E). Photographs were taken at each of time points 0.5 hour (FIG. 2, panels A, F, and K), 1.0 hour (FIG. 2, panels B, G, and L), 3 hours (FIG. 2, panels C, H, and M), 7 hours (FIG. 2, panels D, I, and N) and 12 hours (FIG. 2, panels E, J, and O).

### DETAILED DESCRIPTION

The spine is made up of a series of connected bones called “vertebrae”. The disc is a combination of strong connective tissues that hold one vertebra to the next, and acts as a cushion between the vertebrae. The IVD has a unique structure, having an outer fibrous annulus fibrosus (AF) and an inner gelatinous nucleus pulposus (NP). The AF consists of concentric lamellae rich in collagen fibers, and the NP is an inner gelatinous cushion rich in proteoglycans (PGs). The AF and NP, along with the endplates of the vertebrae above and below, provide the properties of flexibility and resiliency necessary for normal function. The IVD is a large avascular and aneural structure that receives its major nutrition through the endplates via diffusion (Urban, J. P. et al. Nutrition of the intervertebral disk. An in vivo study of solute transport. Clin Orthop, 101-14, 1977).

The extracellular matrix (ECM) of the NP, similarly to that found in articular cartilage is synthesized and maintained throughout adult life by relatively few cells (Jahnke, M. R. & McDevitt, C A. Proteoglycans of the human intervertebral disc. Electrophoretic heterogeneity of the aggregating proteoglycans of the nucleus pulposus. Biochem J 251: 347-56, 1988). Most NP cells are chondrocyte-like (>75%), with a significant number of large notochordal cells present, especially prior to adult life (Maldonado, B. A. et al. Initial characterization of the metabolism of intervertebral disc cells encapsulated in microspheres. J Orthop Res 10: 677-90, 1992). It is not clear if both NP cell types synthesize the large-molecular-weight hydrophilic PG, termed aggrecan, that constitutes the most abundant molecule in the tissue (Aguiar, D. J. et al. Notochordal cells interact with nucleus pulposus cells: regulation of proteoglycan synthesis. Exp Cell Res 246: 129-37, 1999). As in articular cartilage, these aggrecan molecules interact extracellularly with long linear strands of hyaluronan (HA), forming aggregates that become entangled in a fibrillar network made up principally of type II collagen (reviewed in Thonar, E. J. et al. Body fluid markers of cartilage changes in osteoarthritis. Rheum Dis Clin North Am 19: 635-57, 1993). The swelling, fluid and ion-transport properties, as well as the intrinsic mechanical properties of the collagen-aggrecan solid matrix, govern the deformational behavior of the NP. The collagen network gives the tissue tensile strength and hinders expansion of the viscoelastic, under-hydrated, aggrecan molecules that provide compressive stiffness and enable the tissue to undergo reversible deformation.

The AF contains a relatively homogeneous population of chondrocyte-like cells (Maldonado, B. A. et al. Initial characterization of the metabolism of intervertebral disc cells encapsulated in microspheres. J Orthop Res 10: 677-90, 1992) that synthesize a matrix richer in collagen and poorer in PG than cells from the NP. Importantly, some of the cells synthesize PG and collagen molecules not normally found in significant amounts in cartilage (Mayne, R. et al. (ed.). Joint Cartilage degradation 81-108. New York: Marcel Dekker, Inc., 1993; reviewed in Thonar, E. J. et al. Body fluid markers of cartilage changes in osteoarthritis. Rheum Dis Clin North Am 19: 635-57, 1993; Wu, J. J. et al. Type VI collagen of the intervertebral disc. Biochemical and electron-microscopic characterization of the native protein. Biochem J 248: 373-81, 1987). The AF is thus usually classified as a fibrocartilage: it is built for strength rather than to provide reversible deformation. The annulus is not homogenous in that the ratio of proteoglycan to collagen decreases progressively as one moves away from the nucleus. Therefore, it is envisioned that, without being limited by any particular theory or mechanism of action, the cationic molecules could also have an effect on the inner portion of the annulus.

The NP is able to maintain its fluid pressure to balance the high external loads on the IVD because of the abundance of negatively charged PGs. This molecular meshwork of PGs entrapped in a collagen network endows the IVD with both compressive stiffness and tensile strength. Either due to disc degeneration or traumatic injury, a disc can extrude back into the spinal canal, which is known as a herniation, herniated disc, pinched nerve, or bulging disc. The weak spot in a disc is directly under the nerve root, and a herniated disc in this area puts direct pressure on the nerve, which in turn can cause pain, numbness, tingling or weakness of the leg called “sciatica”.

A herniated disc may also cause back pain, although back pain alone (without leg pain) can have many causes other than a herniated disc. Approximately 90% of disc herniations occur at L4/L5 (lumbar segments 4 and 5) or L5/S1 (lumbar segment 5 and sacral segment 1), which causes pain in the L5 nerve or S1 nerve, respectively. L5 nerve impingement from a herniated disc causes weakness in extension of the big toe and potentially in the ankle (foot drop). Numbness and pain can be felt on top of the foot, and the pain may also radiate into the rear. S1 nerve impingement from a herniated disc may cause loss of the ankle reflex and/or weakness in ankle push off (e.g. patients cannot do toe rises). Numbness and pain can radiate down to the sole or to the outside of the foot.

Proteoglycans are macromolecules that are distributed almost everywhere in the body. Their size and structure vary enormously. The basic structure of proteoglycans includes a core protein and at least one, but frequently more (up to tens or hundreds) carbohydrate chains, so called glycosaminoglycans (GAGs). The protein component of proteoglycans is a core protein that directs the biosynthesis of proteoglycans to different molecular constructions and functions. So far, more than 20 genetically different species of core proteins have been identified.

The GAG component includes long unbranched polysaccharides chains composed of repeating disaccharide units, and are called GAGs because one of the two sugars is always an amino sugar e.g. N-acetylglucosamine (GlcNac). GAGs are highly negatively charged due to the presence of sulfate or carboxyl groups or both on many of the sugar residues. The components of disaccharides are glucuronic acid/iduronic acid —N-acetylgalactosamine in chondroitin sulfate and dermatan sulfate, glucuronic acid/iduronic acid —N-acetylglucosamine in heparan sulfate and heparin, and galactose —N-acetylglucosamine in keratan sulfate. Sulfation of glycosaminoglycans in chondroitin sulfate chains is usually regular, one sulfate per disaccharide throughout the chain, while in heparan sulfate chains, sulfation is somewhat irregular, resulting in intensely sulfated and sparsely sulfated regions on a single glycosaminoglycan chain. In addition to glycosaminoglycans, proteoglycans normally have other carbohydrate units including O-linked and N-linked oligosaccharides, as found in other glycosylated proteins.

Biological functions of proteoglycans derive primarily from those of the glycosaminoglycan and protein component of the molecule. Glycosaminoglycans assume extended structures in aqueous solutions because of their strong hydrophilic nature based on their extensive sulfation, a result that is further extended as these molecules are covalently linked to core proteins (a typical example is aggrecan). They hold a large number of water molecules in their molecular domain and as a result occupy a very large hydrodynamic space in solution.

The most studied, prototypical proteoglycan is aggrecan, which constitutes the major component in the NP of the disc as well as articular cartilage and accounts for by far the largest mass among proteoglycans in animal bodies. The molecular mass of aggrecan is about 2500 kDa, and it is structurally a proteoglycan with a core protein of high molecular weight (˜210-250 kDa) encoded by a single gene that is expressed predominantly in cartilaginous tissues. It is highly glycosylated with ˜90% carbohydrate by weight, mainly in the form of 2 types of glycosaminoglycan chains, chondroitin sulfate and keratan sulfate. The core protein has a hyaluronan-binding domain near its amino terminal and forms an enormous supramolecular structure together with hyaluronan and another protein (called the “link protein”). The primary role of aggrecan appears to be a physical one, as it is capable of an osmotic swelling and maintains the high levels of hydration in the extracellular matrix of cartilaginous tissues. The extracellular matrix is comprised of fibril forming collagens, aggrecan and many other important molecules. The fibrillar collagens form a network which has a very high tensile strength, and which entraps the aggrecan molecules. The presence on aggrecan of very large numbers of chondroitin sulfate chains generates an osmotic swelling pressure. Because of this, water makes up a large percentage of the wet weight of the tissue.

During resting, such as sitting down and reading, the osmotic swelling is at a maximum, and is contained only by the collagen network. However, during loading, such as standing up or walking, the weight of the body is supported by the cartilagenous ends of the long bones. In this state the body weight compresses the cartilage, literally squeezing water out. This continues until the osmotic swelling generates a force equal to the compressive force generated by the weight of the body that it supports. When the load is removed, for example by sitting down again, the compressive force is removed, and the cartilage swells to the full extent. The glycosaminoglycan chains attached to the aggrecan core protein bring about this osmotic swelling. Without being limited by any particular theory or mechanism of action, it is envisioned herein that factors that compromise the ability of the glycosaminoglycan chains to exert the osmotic swelling effect will have a severe effect upon the functionality of the tissue.

Polylysine is a lysine polypeptide or homopolymer of lysine. Either or both of the optical isomers of polylysine can be used in the methods, compositions and kits provided herein. The D isomer has the advantage of having long-term resistance to cellular proteases. The L isomer has the advantage of being more rapidly cleared from the subject. Synthetic copolymers of D and L isomers can also be used in the methods, compositions and kits provided herein. At physiological values of pH, polylysine is positively charged. It is commonly used as a coating agent to promote cell adhesion in cell culture applications and as a food additive. Cationic molecules such as polylysine have antimicrobial properties. They are thought to exert these properties by inducing bacterial membrane permeabilization and activating autolytic systems.

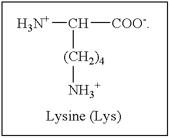

Polylysine has been used as a delivery vehicle for cell targeting moieties, and for MRI contrast agents which are co-transported to specific cells. See Kayyem et al., U.S. Pat. No. 6,232,295 B1. Polylysine conjugates are used as pharmaceuticals agents for treatment of disorders such as autoimmune type and neurodegenerative diseases. See Geffard, U.S. Pat. No. 6,114,388. These agents are administered orally or parenterally, for example by intravenous, intramuscular or subcutaneous administration, or by an implant system permitting subcutaneous profusion. Thonar et al. has used polylysine to prevent proteoglycan loss in cartilage explant culture. See, J. Surg. Res. 56: 302-308, 1994; and Orthopedic Trans, in J. Bone Joint Surg. 13 (2): 301, 1989.

The invention in certain embodiments involves the injection of a cation solution into the extracellular matrix of a tissue in a subject in need of a treatment for disc herniation. In a method provided herein, a percutaneous (performed through the skin) injection of a polylysine solution to the nucleus of a herniated disc is used to reduce intradiscal pressure. The reduction of intradiscal pressure results from noncovalent binding of the polycation (i.e. polylysine) to the anionic components of the extracellular matrix (i.e. the glycosaminoglycan component of proteoglycans). The polycation effectively competes with water for the anionic binding sites. The interaction between water, proteoglycans, and collagen within the disc nucleus is responsible for maintenance of the osmotic pressure. It is the osmotic pressure along with the collagen network that endows the disc with a resistance to deformation under load. Binding of the polycation would prevent water from interacting with the proteoglycan component of the tissue thereby producing an underhydrated or ‘collapsed’ tissue that occupied less space. The resulting ‘collapse’ of the proteoglycan component of disc nucleus leads to a reduction in osmotic pressure, a decrease in herniation and a decrease in pain resulting from the disc herniation.

In another embodiment of the method provided herein, one or more growth factors is covalently bound to a cationic molecule and is again injected percutaneously into the disc to effect disc repair and/or regeneration of a diseased or injured intervertebral disc. Treatment for disc repair and/or regeneration by methods provided herein may occur in various embodiments in conjunction with or separate from treatment of a herniated disc with a cationic solution as described above.

While the use of a monomeric cation solution has the desired outcome, an oligopeptide or polypeptide is also provided. The polycation, polylysine, and other charged polycations can be used. Other polymer cations that may also be useful consist of basic amino acids such as arginine or histidine; dextrans (branched poly-α-D-glucosides); arabinogalactan (water-soluble polysaccharides widely found in plants, fungi and bacteria, the polysaccharides comprising D-galactose and L-arabinose residues); pullulan (extracellular bacterial linear polysaccharide made up of maltotriose produced from starch by Aureobasidium pullulans); cellulose; inulins (a group of naturally occurring fructose-containing oligosaccharides); chitosan (polysaccharide polymers found in fungi, arthropods and marine invertebrates); ornithine (derived from the amino acid arginine); spermidine and spermine (polyamines derived from orhithine); and polyethylenimine (PEI). Positively charged proteins such as lysozyme may also be useful in achieving the desired outcome. An important feature of the injectable material is that it contains a net positive charge. However, in alternative embodiments the cationic compound is complexed with an anionic material, so that the active cationic compound is released slowly after administration and is efficacious over an extended period of time.

Lysine has a molecular weight of 146.19 daltons. Polymers of lysine result from coupling of any number of lysine peptides to form a polymer. The polymer is provided in a range of sizes, commonly from 2 to greater than 300 kDa. In various embodiments of the present invention, polylysine can be either a linear or branched polypeptide administered as a solution containing single or multiple molecular weight polypeptide chains. When poly amino acids such as poly-lysine, -arginine, or -histidine are used, preferred sizes range from about 100 to 300 kDa. The polycation used should be of sufficient size to bind to more than one proteoglycan molecule.

Once the cationic agent has functioned efficaciously, e.g., tissue has collapsed, the effect of the cation on the structure is not necessarily permanent. Over time water will slowly replace or compete for anionic binding sites and rehydrate the tissue. There are several features of the invention that aid in extending the half-life of the desired effect. As polylysine turnover begins post injection, rehydration occurs directionally, i.e., from the outside-in. The net result is a swelling of the outermost areas of the treatment site. This swelling has a negative effect on the further release of bound polylysine, as the polylysine must traverse through tissue with a higher net osmotic pressure. Preliminary results in examples herein indicate that a reduction in the Disc Height Index (DHI, a measurement of the height of the disc) was achieved for at least 6 months post treatment. Polylysine of different molecular weights can have different half-lives post injection. This is an important feature of the invention as it would be advantageous to be able to predict the duration of an effect.

Just as there is a need to provide relief from the symptoms of intervertebral disc herniation, there is also a need to enhance the stability and repair of the NP and AF. In another embodiment of the invention provided herein, one or more growth factors can be covalently bound to a cationic molecule and these also can be injected percutaneously into the disc to effect disc repair and/or regeneration of a diseased or injured intervertebral disc. Treatment for disc repair and/or regeneration may occur in conjunction with or separate from treatment of a herniated disc with a cationic solution as described above.

One or more covalently bound growth factors well known in the art of cell biology, including but not limited to, bone morphogenesis proteins BMP-2 and BMP-7, insulin-like growth factor (IGF), transforming growth factor (TGF), growth differentiation factor GDF-5, and platelet derived growth factor (PDGF) are, in various embodiments, co-administered with or are covalently bound to the polycation compound therapeutic agent herein, to effect a change in matrix synthesis and/or degradation.

In addition, enzyme digestion, e.g., of protruding tissue, can be used in combination with the therapeutic methods and compositions herein. Suitable methods and compositions for combining with those provided herein such as chemonucleolysis, using a glycosidase such as chondroitinase such as chondroitinase A, or chondroitinase B, or chondroitinase C, or chondroitinase ABC, are described by Masuda et al., U.S. patent application No. 2004/0033221 A1.

Conversely, compositions and methods herein can be combined with inhibitors of matrix degradation such as matrix metalloproteinase inhibitors may also be useful in preventing disease progression. One or more of these molecules is targeted to the extracellular matrix of the disc using one or more of a cationic molecule. The benefits include both a reduction in osmotic pressure, and a specific targeting of the molecules. Specific targeting of the anabolic and catabolic factors through the noncovalent bonds formed between cation (polylysine) and proteoglycan allows for decreased concentrations of growth factors or metalloproteinase inhibitors to be used yet still effect clinically significant result. For example, these factors can also increase the half-lives of these molecules in vivo.

The treatment methods of the invention herein are used for treating a subject which is a mammal, such as a mouse, rat, rabbit, dog, horse, or a primate such as a monkey, chimpanzee or human that has a condition requiring the treatment method.

A compound useful in methods and kits the present invention can be formulated as a pharmaceutical composition. Such a composition can then be administered parenterally, for example, in dosage unit formulations containing conventional nontoxic pharmaceutically acceptable carriers and vehicles as desired. The term parenteral as used herein includes intervertebral injections, or local infusion techniques. Formulation of drugs is discussed in, for example, Hoover, John E. (ed.), Remington's Pharmaceutical Sciences (18th Edition), Mack Publishing Co., Easton, Pa., 1990 and Liberman, H. A. and Lachman, L., Eds., Pharmaceutical Dosage Forms, Marcel Decker, New York, N.Y., 1980. As discussed elsewhere herein, the administration of a cationic polymer, alone or in combination with another agent such as a growth factor is typically by direct local injection or by local infusion into the intervertebral disk space.

Injectable preparations, for example, sterile injectable aqueous or oleaginous suspensions can be formulated according to the known art using suitable dispersing or wetting agents and suspending agents. The sterile injectable preparation can also be a sterile injectable solution or suspension in a nontoxic parenterally acceptable diluent or solvent, for example, as a solution in 1,3-butanediol. The suspension provides for a delayed release formulation. Among the acceptable vehicles and solvents that can be employed are water, Ringer's solution, and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid find use in the preparation of injectables. Dimethyl acetamide, surfactants including ionic and non-ionic detergents, polyethylene glycols can be used. Mixtures of solvents and wetting agents such as those discussed above are also useful.

For therapeutic purposes, formulations for administration can be in the form of aqueous or non-aqueous isotonic sterile injection solutions or suspensions. These solutions and suspensions can be prepared from sterile powders or granules having one or more of the carriers or diluents used in formulations for oral administration, as is well known in the art. The compounds can be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

A compound useful in the present invention can also be formulated into liposomes, as discussed in Hoover, John E. (ed.), Remington's Pharmaceutical Sciences (18.sup.th Edition), Mack Publishing Co., Easton, Pa., 1990, p. 1691. Liposomes are formed by dispersing phospholipids in an aqueous medium. Water- or lipid-soluble substances can be entrapped in the aqueous space within a liposome, or within the lipid bilayers of the liposome, respectively. Thus, a growth factor can be formulated into liposomes for use in a method of the invention using techniques that are well known in the art.

As noted above, the amount of the active ingredient that can be combined with the carrier materials to produce a single dosage form varies, depending upon the mammalian subject to be treated, and the particular mode of administration as in known to one of skill in the art of pharmaceutical sciences.

The present invention in another embodiment includes a kit comprising a cationic polymer such as a polylysine. The kit contains the polymer, alone or in a pharmaceutical buffer, optionally with a further agent such as a growth factor, or an enzyme or enzyme inhibitor. The kit is packaged in a conventional manner, as is well known in the art.

The kits of the invention preferably comprise a vessel containing a cationic polymer and a vessel containing another agent such as a growth factor. Such a vessel can be a glass vial or container, a plastic vial or container, or other suitable container, as is well known in the art. In another preferred embodiment, the kit comprises a vessel containing a cationic polymer and a growth factor. Such a kit is especially suited for simultaneous administration of the polymer alone or in combination with another agent such as a growth factor according to a method of the invention.

Any of the above-described kits optionally includes instructions for using the polymer. For example, the instructions provide details on using the components of the kit in a method of the present invention.

### Pharmaceutical Compositions

In another aspect, the present invention provides a composition, e.g., a pharmaceutical composition, containing one or a combination of cationic compounds, or polymers thereof, of the present invention, formulated together with a pharmaceutically acceptable carrier. Such compositions may include one or a combination of (e.g., two or more different) cations, or polymers thereof such as polylysine. For example, a pharmaceutical composition of the invention can comprise a combination of cations, such as poly-L-lysine and poly-D-lysine, or poly-L-lysine compounds of different lengths.

Pharmaceutical compositions of the invention can in various embodiments of methods herein, be administered in combination therapy, i.e., combined with other agents. For example, the combination therapy can include a cationic compound of the present invention combined with at least one anti-inflammatory or anti-microbial agent. Examples of therapeutic agents that can be used in combination therapy are described in greater detail below in the section on uses of the antibodies of the invention.

As used herein, “pharmaceutically acceptable carrier” includes any and all solvents, dispersion media, coatings, antibacterial and antifungal agents, isotonic and absorption delaying agents, and the like that are physiologically compatible. The carrier should be suitable for intravenous, spinal such as intravertebral, or intramuscular, subcutaneous, parenteral, or epidermal administration (e.g., by injection or infusion). Depending on the route of administration, the active compound, i.e., cationic compound or polymer, may be coated in a material to protect the compound from the action of acids and other natural conditions that may inactivate the compound.

The pharmaceutical compounds of the invention may include one or more pharmaceutically acceptable salts. A “pharmaceutically acceptable salt” refers to a salt that retains the desired biological activity of the parent compound and does not impart any undesired toxicological effects (see e.g., Berge, S. M., et al., 1977 J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and di-carboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A pharmaceutical composition of the invention also may include a pharmaceutically acceptable anti-oxidant. Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents such as sugars, sodium chloride, and the like into the compositions. In addition, prolonged absorption of the injectable pharmaceutical form may be brought about by the inclusion of agents which delay absorption, such as aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, one can include isotonic agents, for example, sugars, polyalcohols such as mannitol, sorbitol, or sodium chloride in the composition. While rapid absorption is in general contemplated as a preferred result of administration, prolonged absorption of the injectable compositions can be brought about by including in the composition an agent that delays absorption, for example, monostearate salts, and/or gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the methods of preparation are vacuum drying and freeze-drying (lyophilization) that yield a powder of the active ingredient plus any additional desired ingredient from a previously sterile-filtered solution thereof.

The amount of active ingredient that can be combined with a carrier material to produce a single dosage form will vary depending upon the subject being treated, and the particular mode of administration. The amount of active ingredient that can be combined with a carrier material to produce a single dosage form will generally be that amount of the composition which produces a therapeutic effect. Generally, out of one hundred percent, this amount will range from about 0.01 percent to about ninety-nine percent of active ingredient, from about 0.1 percent to about 70 percent, or from about 1 percent to about 30 percent of active ingredient in combination with a pharmaceutically acceptable carrier.

Dosage regimens are adjusted to provide the optimum desired response (e.g., a therapeutic response that can be rapid and even result in an immediate reduction in back pain). For example, a single bolus may be administered, several divided doses may be administered over time or the dose may be proportionally reduced or increased as indicated by the exigencies of therapeutic situation. It is especially advantageous to formulate intravertebral compositions in unit dosage form for ease of administration and uniformity of dosage. Unit dosage form as used herein refers to physically discrete units suited as unitary or single dosages for the subject to be treated; each unit contains a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of a range of symptoms among a plurality of individual subjects.

For administration of the cationic compound, the dosage ranges from about 0.0001 to 100 mg/kg, and more usually 0.01 to 5 mg/kg, of the subject body weight. For example dosages can be 0.3 mg/kg body weight, 1 mg/kg body weight, 3 mg/kg body weight, 5 mg/kg body weight or 10 mg/kg body weight or within the range of 1-10 mg/kg. An exemplary treatment regimen entails administration once per week, once every two weeks, once every three weeks, once every four weeks, once a month, once every 3 months or once every three months to 6 months. Dosage regimens for a cationic compound of the invention include about 1 mg/kg body weight or about 3 mg/kg body weight by intravertebral administration, with the cationic compound being given using one of the following dosing schedules: every four weeks for six dosages, then every three months; every three weeks; about 3 mg/kg body weight once followed by about 1 mg/kg body weight every three weeks.

In some methods, two or more cationic compounds with different rates of permeation and/or clearance are administered simultaneously or sequentially in a protocol or regimen, in which case the dosage of each compound administered falls within the ranges indicated. Cationic compound is usually administered on multiple occasions, although for certain individuals a single treatment can remediate the condition fully. Intervals between single dosages can be, for example, weekly, monthly, every three months or yearly. Intervals can also be irregular as indicated by measuring intravertebral height, or merely symptoms in the patient. In some methods, dosage is adjusted to achieve a condition of pain remediation.

Alternatively, the cationic compound can be administered as a sustained release formulation, in which case less frequent administration is required. Dosage and frequency vary depending on the dwell time or half-life presence of the compound in the disc in the patient. In general, longer polymers show the longest half-life, followed by polymers of intermediate length. The dosage and frequency of administration can vary depending on whether the treatment is prophylactic or therapeutic. In prophylactic applications, a relatively low dosage is administered at relatively infrequent intervals over a long period of time. Some patients continue to receive treatment for the duration of their lives. In therapeutic applications, a relatively high dosage at relatively short intervals may be indicated until progression of the disease is reduced or terminated or until the patient shows partial or complete amelioration of symptoms of disease. Thereafter, the patient can be administered a prophylactic regime.

Actual dosage levels of the active ingredients in the pharmaceutical compositions of the present invention may be varied so as to obtain an amount of the active ingredient that is effective to achieve the desired therapeutic response for a particular patient, composition, and mode of administration. The selected dosage level will depend upon a variety of pharmacokinetic factors including the activity of the particular compositions of the present invention employed, or the ester, salt or amide thereof, the route of administration, the time of administration, the rate of clearance from the disc of the particular compound being employed, the duration of the treatment, co-administration of other drugs, compounds and/or materials used in combination with the particular compositions employed, the age, sex, weight, condition, general health and prior medical history of the patient being treated, and like factors well known in the medical arts.

A “therapeutically effective dosage” of the cationic compound of the invention can results in a decrease in severity of disease symptoms, an increase in frequency and duration of disease symptom-free periods, or a prevention of impairment or disability due to the disease affliction.

A composition of the present invention can be administered by one or more routes of administration using one or more of a variety of methods known in the art. As will be appreciated by the skilled artisan, the route and/or mode of administration will vary depending upon the desired results. A suitable route of administration for cationic compounds of the invention is intravertebral, although intravenous, intramuscular, intradermal, intraperitoneal, subcutaneous, spinal and other parenteral routes of administration are within the scope of the invention, for example by injection or infusion. The phrase “parenteral administration” as used herein means modes of administration other than enteral and topical administration, usually by injection, and includes, without limitation, intravertebral, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, and particularly includes injection or infusion directly into a joint of the spine.

The active compounds can be prepared with carriers that will protect the compound against rapid release, such as a controlled release formulation, including implants, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

Therapeutic compositions can be administered with medical devices known in the art. For example, in one embodiment, a therapeutic composition of the invention can be administered with a needleless hypodermic injection device, such as the devices shown in U.S. patents having numbers: U.S. Pat. No. 5,399,163; 5,383,851; 5,312,335; 5,064,413; 4,941,880; 4,790,824 or 4,596,556. Examples of well known implants and modules useful in the present invention include: U.S. Pat. No. 4,487,603, which shows an implantable micro-infusion pump for dispensing medication at a controlled rate; U.S. Pat. No. 4,486,194, which shows a therapeutic device for administering medicaments through the skin; U.S. Pat. No. 4,447,233, which shows a medication infusion pump for delivering medication at a precise infusion rate; U.S. Pat. No. 4,447,224, which shows a variable flow implantable infusion apparatus for continuous drug delivery; U.S. Pat. No. 4,439,196, which shows an osmotic drug delivery system having multi-chamber compartments; and U.S. Pat. No. 4,475,196, which shows an osmotic drug delivery system. These patents and other references cited are hereby incorporated herein by reference. Many other such implants, delivery systems, and modules are known to those skilled in the art.

For methods of manufacturing liposomes, see, e.g., U.S. Pat. Nos. 4,522,811; 5,374,548; and 5,399,331. The liposomes may comprise one or more moieties which are selectively transported into specific cells or organs such as the discs, thus enhance targeted drug delivery (see, e.g., V. V. Ranade, 1989 J. Cline Pharmacol. 29:685). Exemplary targeting moieties include folate or biotin (see, e.g., U.S. Pat. No. 5,416,016 to Low et al.); mannosides (Umezawa et al., 1988 Biochem. Biophys. Res. Commun. 153:1038); antibodies (P. G. Bloeman et al., 1995 FEBS Lett. 357:140; M. Owais et al., 1995 Antimicrob. Agents Chemother. 39:180); surfactant protein A receptor (Briscoe et al., 1995 Am. J. Physiol. 1233:134); p 120 (Schreier et al., 1994 J. Biol. Chem. 269:9090); see also K. Keinanen; M. L. Laukkanen, 1994 FEBS Lett. 346:123; J. J. Killion; I. J. Fidler, 1994 Immunomethods 4:273.

### Uses and Methods of the Invention

The term “subject” as used herein in intended to include human and non-human animals. Non-human animals includes all vertebrates, e.g., mammals and non-mammals, such as non-human primates, sheep, dogs, cats, cows, horses, chickens, other warm blooded vertebrates such as birds, and cold-blooded vertebrates such as amphibians and reptiles. The methods are particularly suitable for treating human patients having a disorder associated with aberrant vertebral disc morphology. When the cationic compounds are administered together with another agent, the two can be administered in either order or simultaneously.

Also within the scope of the invention are kits including the compositions (e.g., cationic compounds and polymers thereof) of the invention and instructions for use. The kit can further contain a least one additional agent, or one or more additional cationic compounds of the invention (e.g., a compound having a polymer composition of length or component monomers for longer lasting activity). Kits typically include a label indicating the contents and the intended use of the contents of the kit. The term label includes any writing, or recorded material supplied on or with the kit, or which otherwise accompanies the kit, such as instructions for use.

The invention having been fully described, it is further illustrated by the following examples and claims, which are illustrative only and are not meant to be further limiting. Those skilled in the art will recognize or be able to ascertain using no more than routine experimentation, numerous equivalents to the specific procedures described herein. Such equivalents are within the scope of the present invention and claims. The contents of all references, including issued patents and published patent applications, cited throughout this application are hereby incorporated by reference.

The following examples are offered to further illustrate, but not limit the present invention.

### EXAMPLES

### Materials and Methods

Cell Culture: Lumbar intervertebral disks (IVDs) were aseptically dissected from the spine of New Zealand white rabbits after euthanasia. The NP and AF were separated by blunt dissection as necessary and separately pooled.

Statistical Analyses: Statistical analyses were performed by one way ANOVA with Fisher's PLSD test as a post hoc test.

### Example 1

### Effect of Polylysine on Disc Height

Rabbit discs (six animals) were injected with 30-70 kDa polylysine into discs L4/5 and L2/3. As controls, the L3/4 discs were untreated in each of the animals.

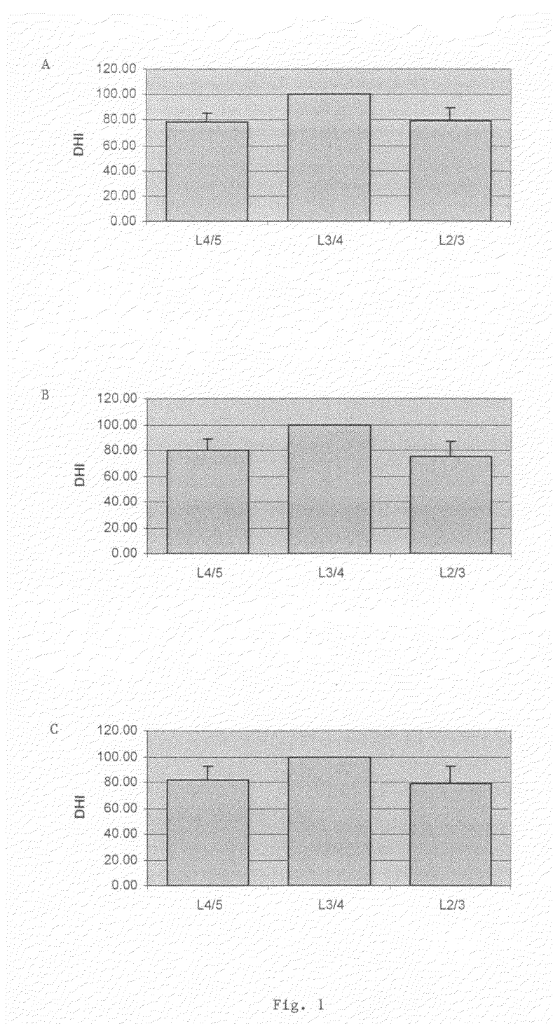

Disc height was measured at each of 2, 4 and 6 weeks after injection, and the normalized percentages of starting disc heights observed are shown in FIG. 1. Disc height was reduced by the treatment to about 80% of the initial values, and the decrease of 20% was maintained for the full 6-week observation period. The data are shown in Table 1.

These data indicate that the polylysine successfully placed the discs in a condition in which herniation is much less likely, and in which repair and regeneration are more likely.

TABLE 1
Disc height index of treated and control rabbit lumbar
intervertebral discs

| | L4/5 DHI | L3/4 DHI | L2/3 DHI |
|---|---|---|---|
| 2 weeks | | | |
| 1 | 72.34 | 100 | 91.98 |
| 2 | 81.34 | 100 | 80.01 |
| 3 | 73.56 | 100 | 89.04 |
| 4 | 90.03 | 100 | 77.13 |
| 5 | 77.07 | 100 | 68.43 |
| 6 | 77.69 | 100 | 70.29 |
| Average | 78.67 | 100 | 79.48 |
| Standard deviation | 6.4149 | 0 | 9.5917 |
| Standard deviation (total) | 7.791082 | | |
| Count | 12 | | |
| Standard error | 2.25 | | |
| 4 weeks | | | |
| 1 | 79.68 | 100 | 79.11 |
| 2 | 93.65 | 100 | 93.42 |
| 3 | 66.64 | 100 | 80.98 |
| 4 | 79.43 | 100 | 66.08 |
| 5 | 73.86 | 100 | 63.83 |
| 6 | 84.63 | 100 | 65.74 |
| Average | 79.65 | 100 | 74.86 |
| Standard deviation | 9.2042 | 0 | 11.6788 |
| Standard deviation (total) | 10.33254 | | |
| Count | 12 | | |
| Standard error | 2.98 | | |
| 6 weeks | | | |
| 1 | 87.13 | 100 | 78.77 |
| 2 | 96.43 | 100 | 93.30 |
| 3 | 81.53 | 100 | 96.63 |
| 4 | 83.27 | 100 | 65.59 |
| 5 | 61.60 | 100 | 73.66 |
| 6 | 79.46 | 100 | 69.09 |
| Average | 81.57 | 100 | 79.51 |
| Standard deviation | 11.4704 | 0 | 12.8076 |
| Standard deviation (total) | 11.6417 | | |
| Count | 12 | | |
| Standard error | 3.36 | | |

### Example 2

### Distribution Kinetics of Polylysine into Cartilage

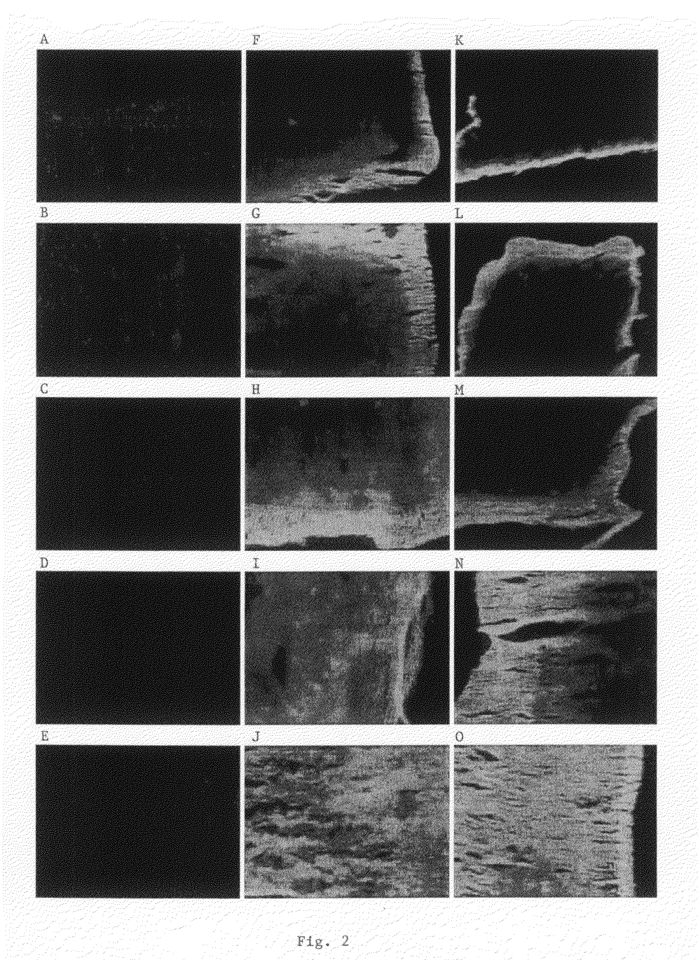

Polylysine was observed to enter cartilage explant tissues during incubation, as shown in FIG. 2 using polylysine polymers labeled with fluorescein isothiocyanate (FITC). Tissue was incubated as explants submerged in solutions of polylysine as shown in FIG. 2. After a predetermined time, tissue was washed, frozen, and 10 micron sections were produced using a cryostat.

After 30 minutes of incubation, the polylysine was seen to have permeated the edges of the explants (FIG. 2 panels F and K compared to control with no polylysine, panel A). By one hour, additional fluorescence became visible in the interior of the explant (FIG. 2 panels G and L compared to control with no polylysine, panel B), and more had permeated by three hours (FIG. 2 panels H and M compared to control with no polylysine, panel C).

These data show that relief from pain associated with herniated disc can potentially be achieved rapidly following treatment of discs with polylysine.

### Example 3

### Control of Permeation with Polymer Length

Rapidity of permeation of cartilage was enhanced by treatment with polylysine having a shorter chain length (15-30 kDa, about 102-204 monomers of lysine) as shown in FIG. 2, panels F, G, H, I, and J, compared to treatment with a longer chain length (30-70 kDa, up to about 479 monomers) as shown in FIG. 2, panels K, L, M, N, and O.

Using the shorter cationic polymer, permeation was observed to have achieved maximal levels, i.e., was completed, within 3-7 hours (FIG. 2 panel H compared to panel M), compared to within 7-12 hours for the longer polymer to achieve maximal levels (FIG. 2, panels N and O compared to panels I and J).

A polymer having a more rapid rate of permeation is useful as a potential therapeutic agent for remediation of acute pain associated with disc protrusion, such as sciatica, because the cartilage condensation and decrease in intervertebral distance would retract extruded material.

In addition, the longer polymers offer the possibility of a longer-acting therapeutic agent.

What is claimed is:

1. A pharmaceutical composition for remediating a cartilage disease comprising an effective dose of a cationic compound in an effective dose, and a pharmaceutically acceptable salt or buffer.

2. The composition according to claim 1, wherein the compound is a polymer of a cationic monomer.

3. The composition according to claim 1, wherein the compound is a polymer of an amino acid.

4. The composition according to claim 3, wherein the amino acid is at least one selected from a group consisting of D-lysine, L-lysine, D-arginine, L-arginine, D-histidine, and L-histidine.

5. The composition according to claim 1, wherein the compound is at least one selected from the group consisting of a dextran, an arabinogalactan, a pullulan, a cellulose, an inulin, a chitosan, an ornithine polymer, a spermine polymer, a spermidine polymer, and polyethylenimine.

6. The composition according to claim 5, wherein the dextran is a branched poly-α-D-glucoside.

7. The composition according to claim 5, wherein the arabinogalactan comprises D-galactose and L-arabinose.

8. The composition according to claim 5, wherein the arabinogalactan is produced by a plant, a fungus, or a bacterium.

9. The composition according to claim 5, wherein the pullulan comprises maltotriose.

10. The composition according to claim 9, wherein the pullulan is produced by Aureobasidium pullulans.

11. The composition according to claim 5, wherein the inulin comprises fructosyl oligosaccharides.

12. The composition according to claim 5, wherein the chitosan is produced by at least one selected from the group consisting of: a fungus, an arthropod, or a marine invertebrate.

13. The composition according to claim 1, wherein the cationic compound is a protein.

14. The composition according to claim 13, wherein the protein is lysozyme.

15. The composition according to claim 3, wherein the polymer is at least about 2 kDa in average size.

16. The composition according to claim 15, wherein the polymer is at least about 300 kDa in size.

17. The composition according to claim 3, wherein the polymer is a polylysine, and the polylysine is about 100 kDa to about 300 kDa in size.

18. The composition according to claim 3, wherein the polymer is of sufficient size to bind to a plurality of proteoglycan molecules.

19. The composition according to claim 1, comprising a plurality of polycationic compounds selected for varying molecular weights, wherein a half-life of the compound in a cartilage is a function of the molecular weight.

20. The composition according to claim 1, further comprising a growth factor or an enzyme inhibitor such as a matrix metalloproteinase inhibitor.

21. The composition according to claim 20, wherein the growth factor is at least one selected from the group of a bone morphogenesis protein such as BMP-2 and BMP-7, an insulin like growth factor (IGF), a transforming growth factor (TGF), a growth differentiation factor (GDF) such as GDF-5, and a platelet-derived growth factor (PDGF).

22. The composition according to claim 20, wherein the growth factor or enzyme inhibitor is covalently bound to the compound.

23. A method for remediating a cartilage disease in a subject, the method comprising:
contacting the cartilage with a composition comprising a cationic compound; and
observing remediation of the cartilage disease in the subject.

24. The method according to claim 23, wherein the cartilage is an intervertebral disc.

25. The method according to claim 24, wherein the disc is herniated.

26. The method according to claim 23, wherein contacting the cartilage is injecting a solution of the composition.

27. The method according to claim 26, wherein injecting is percutaneous.

28. The method according to claim 23, wherein remediation is observing a reduction in at least one of disc height, back pain, sciatica, pinched nerve, extrusion, herniation, foot drop, and loss of ankle reflex.

29. The method according to claim 23, wherein the remediation is disc regeneration.

30. The method according to claim 23, wherein the compound is polylysine.

31. The method according to claim 30, wherein the polylysine comprises L-lysine.

32. The method according to claim 30, wherein the polylysine is a D- and L-heteropolymer.

33. The method according to claim 24, wherein the compound is at least one selected from the group consisting of a dextran, an arabinogalactan, a pullulan, a cellulose, an inulin, a chitosan, an alginate, an ornithine polymer, a spermine polymer, a spermidine polymer, and polyethylenimine.

34. A kit for treating a herniated disc, comprising a cationic compound, a container, and instructions for use in treating a herniated disc.

35. The kit according to claim 34, wherein the compound is present in a unit dose for administering by hypodermic intravertebral injection.
